(19) United States
(12) Patent Application Publication
(10) Pub. No.: US 2014/0039387 A1
(43) Pub. Date: Feb. 6, 2014

(54) GUIDE TUBE FOR MICROSURGICAL INSTRUMENTS

(71) Applicants: Seung Jong KIM, Seoul (KR); Kyung Geune OH, Seoul (KR); Seung Bin KIM, Seoul (KR); Sung Chul KANG, Seoul (KR)

(72) Inventors: Seung Jong KIM, Seoul (KR); Kyung Geune OH, Seoul (KR); Seung Bin KIM, Seoul (KR); Sung Chul KANG, Seoul (KR)

(73) Assignee: KOREA INSTITUTE OF SCIENCE AND TECHNOLOGY, Seoul (KR)

(21) Appl. No.: 13/957,741

(22) Filed: Aug. 2, 2013

(30) Foreign Application Priority Data
Aug. 3, 2012 (KR) 10-2012-0085082

(51) Int. Cl. A61M 25/01

(52) U.S. Cl. CPC A61M 25/0147
USPC 604/95.04

(57) ABSTRACT

A guide tube for microsurgical instruments has a linear tube having a hollow formed in the length direction thereof, a curved tube mounted to the front end of the linear tube and having unit bodies connected to allow bending, an elastic body for giving an elastic force so that a gap between the unit bodies increases, and a tension control unit mounted to the rear end of the linear tube to bend the curved tube by controlling tensions of a plurality of wires extending from the curved tube, wherein the tension control unit controls the tensions of the wires in a state where the curved tube is curved so that the rigidity of the curved tube increases to support a surgical instrument located in the linear tube and the curved tube.

### CROSS-REFERENCE TO RELATED APPLICATION

This application claims priority to Korean Patent Application No. 10-2012-0085082, filed on Aug. 3, 2012, and all the benefits accruing therefrom under 35 U.S.C. §119, the contents of which in its entirety are herein incorporated by reference.

### BACKGROUND

1. Field

The present disclosure relates to a guide tube for active surgical instruments, and more particularly, to a guide tube configured to allow bending of a terminal of a surgical instrument to be used for the minimally invasive surgery of a neurosurgery and also allow the rigidity to change so that a specific shape may be maintained.

2. Description of the Related Art

In the field of neurosurgery, the intracranial surgery using the minimally invasive method such as a transsphenoidal approach frequently demands minute manipulations in a deep and narrow work space. Therefore, the approach to the lesion is often restricted without a specially developed instrument.

In particular, even though the spot of the lesion is visually checked by using a fixed-angle endoscopy or the like, direct approach and manipulation is impossible in many cases by using existing linear surgical instruments. If this problem occurs, tissues around the lesion are used to draw the lesion to a work space which may be manipulated, or the craniotomy is performed to entirely expose the lesion before the surgery.

However, the former method takes a long operation time and greatly depends on the experiences and skill level of an operator, and the later method is a major surgery consuming about 10 hours or more, which frequently causes postoperative complications, lengthens the recovery period and remains great surgical scars.

In this regard, instruments used for the single port laparoscopic surgery implement the curve of their terminals by using wires or the like for the manipulation in a narrow work space, but their diameter is too great to be used for microsurgeries of the neurosurgery.

Therefore, it is urgent in the field of neurosurgery minimally invasive surgery to develop a guide tube which may have a small diameter to be available in a narrow work space such as in the transsphenoidal approach, freely bend to a desired direction, maintain the rigidity as desired for the work in a bending state, and allow the lesion removing work as an existing insertion-type surgical instrument.

### SUMMARY

The present disclosure is directed to providing a guide tube for microsurgical instruments, which is configured to freely bend at a terminal thereof and allow the rigidity to change so that the rigidity may be maintained in a bending state.

In one aspect, there is provided a guide tube for microsurgical instruments, which includes: a linear tube having a hollow formed in the length direction thereof; a curved tube mounted to the front end of the linear tube and having unit bodies connected to allow bending; an elastic body for giving an elastic force so that a gap between the unit bodies increases; and a tension control unit mounted to the rear end of the linear tube to bend the curved tube by controlling tensions of a plurality of wires extending from the curved tube, wherein the tension control unit controls the tensions of the wires in a state where the curved tube is curved so that the rigidity of the curved tube increases to support a surgical instrument located in the linear tube and the curved tube.

According to an embodiment of the present disclosure, the unit body of the curved tube may have a hemispherical ball formed at a front end thereof and a socket with a hemispherical groove formed at a rear end thereof, and the ball of the unit body may be matched with and connected to a socket of another unit body.

According to an embodiment of the present disclosure, a flange may be formed at the outer circumference of the unit body, and a wire having one end fixed to the unit body located at the front end of the curved tube may extend rearwards through a hole formed in a flange of another unit body.

According to an embodiment of the present disclosure, the elastic body may be a coil spring, which is located at the inside of the wire passing through the hole of the flange while surrounding the outer circumference of the unit body, and both ends of the coil spring may come into contact with flanges of unit bodies located in the front and rear direction.

According to an embodiment of the present disclosure, the front end of the linear tube may have a hemispherical ball structure, which is matched with a socket of a unit body located at the rear end of the curved tube.

According to an embodiment of the present disclosure, a flange may be formed at the outer circumference of the linear tube, and a wire extending rearwards from the curved tube may extend to the tension control unit through a hole formed in the flange of the linear tube.

According to an embodiment of the present disclosure, a plurality of wires disposed at equal angles along the circumferences of the linear tube and the curved tube may extend in the length direction of the curved tube and the linear tube and be connected to the tension control unit.

According to an embodiment of the present disclosure, there may be provided a pair of tension control units, which contrarily control tensions of the wires extending to the rear of the linear tube so that the curved tube is bent.

According to an embodiment of the present disclosure, there may be provided a pair of tension control units, which increase or decrease tensions of the wires extending to the rear of the linear tube in a lump so that the rigidity of the curved tube is controlled.

According to an embodiment of the present disclosure, the tension control unit may include a steering dial connected to the rear ends of the wires to contrarily control tensions of the wires by turning.

According to an embodiment of the present disclosure, the tension control unit may include a cam dial for locking the curved tube to maintain a curved state by increasing a gap between the wires extending rearwards and increasing tensions of the wires or releasing the locking of the curved tube so that the curved tube is capable of bending by decreasing the gap between the wires and decreasing the tensions thereof.

According to an embodiment of the present disclosure, there may be provided two wires, which are respectively located at both sides of the linear tube and the curved tube, and tensions of two wires are contrarily controlled by turning the steering dial connected to the rear ends of the two wires.

According to an embodiment of the present disclosure, there may be provided two wires, which are respectively located at both sides of the linear tube and the curved tube, and a cam of the cam dial located between two wires is turned to increase a gap between the two wires and increase tensions of the two wires or decrease the gap between the two wires and decrease tensions of the two wires.

According to an embodiment of the present disclosure, there may be provided four wires, which are located at equal angles along the circumferences of the linear tube and the curved tube, and tensions of two wires are contrarily controlled by turning a first steering dial connected to the rear ends of the two wires and tensions of the other two wires are contrarily controlled by turning a second steering dial connected to the rear ends of the other two wires.

According to an embodiment of the present disclosure, there may be provided four wires, which are located at equal angles along the circumferences of the linear tube and the curved tube, and a cam of a first cam dial located between two wires is turned to increase a gap between the two wires and increase tensions of the two wires or decrease the gap between the two wires and decrease tensions of the two wires, and a cam of a second cam dial located between the other two wires may be turned to increase a gap between the other two wires and increase tensions of the other two wires or decrease the gap between the other two wires and decrease tensions of the other two wires.

According to an embodiment of the present disclosure, an insert hole may be formed in the tension control unit so that a surgical instrument is inserted into the hollow of the linear tube and the hollow of the curved tube.

As described above, the guide tube of the present disclosure may freely bend at its terminal so that a microsurgical instrument may approach the lesion and allow a surgical operation, and so the microsurgical instrument may be safely guided to the lesion.

In addition, the guide tube of the present disclosure may have enhanced strength to maintain a linear or bending state, and so even though a microsurgical instrument enters the guide tube, the guide tube may maintain the posture of the microsurgical instrument so that the microsurgical instrument may safely approach into the lesion.

In addition, the guide tube the present disclosure may freely bend and allow simple operation and processing since a connection portion of cylinders is configured with a ball and a socket.

### BRIEF DESCRIPTION OF THE DRAWINGS

The above and other aspects, features and advantages of the disclosed exemplary embodiments will be more apparent from the following detailed description taken in conjunction with the accompanying drawings in which:

FIG. 1 is a perspective view showing a guide tube for microsurgical instruments according to the present disclosure;

FIG. 2 is an exploded perspective view showing a terminal of the guide tube of FIG. 1;

FIG. 3 is a cross-sectional view showing the guide tube of FIG. 1;

FIG. 4 is a detailed diagram showing a coupling relation among a coil spring, a flange and a wire hole employed in the guide tube of FIG. 1;

FIG. 5 is a cross-sectional view showing the terminal of the guide tube of FIG. 1 which is in a bending state;

FIG. 6 is a conceptual diagram showing a microsurgical instrument inserted into the guide tube of FIG. 5;

FIG. 7 is a detailed diagram showing a tension control unit employed in the guide tube of FIG. 1; and

FIGS. 8A and 8B are conceptual diagrams showing a relation of controlling a curve and rigidity of the curved tube by using the tension control unit of FIG. 7.

### DETAILED DESCRIPTION

Hereinafter, a guide tube for microsurgical instruments according to exemplary embodiments of the present disclosure will be described in detail with reference to the accompanying drawings.

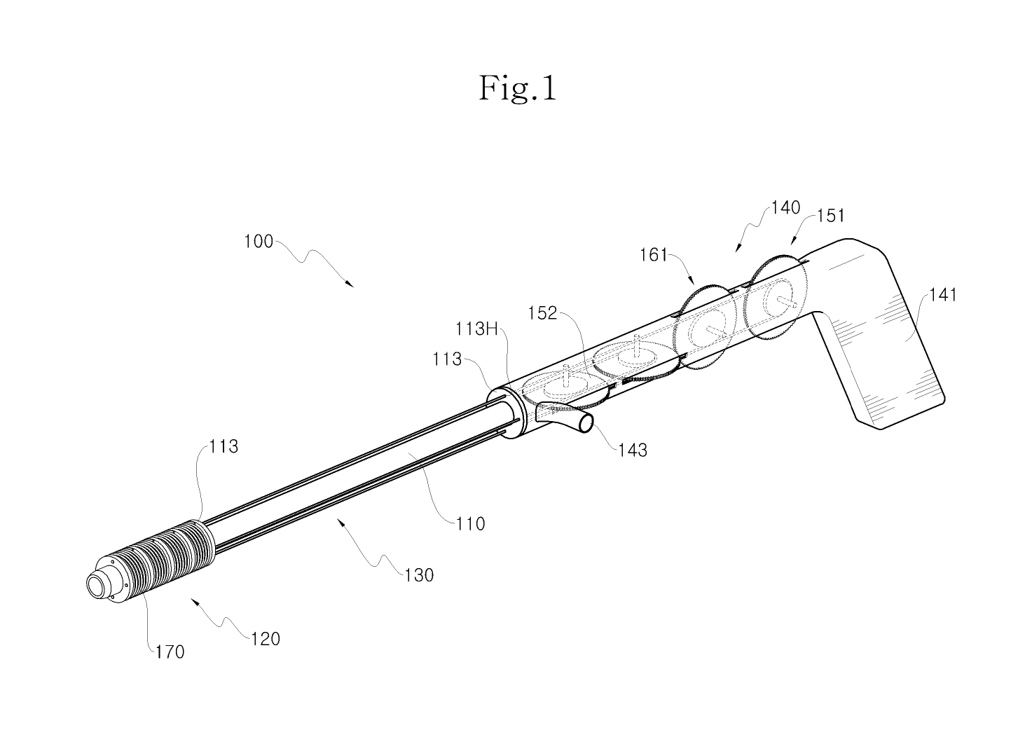

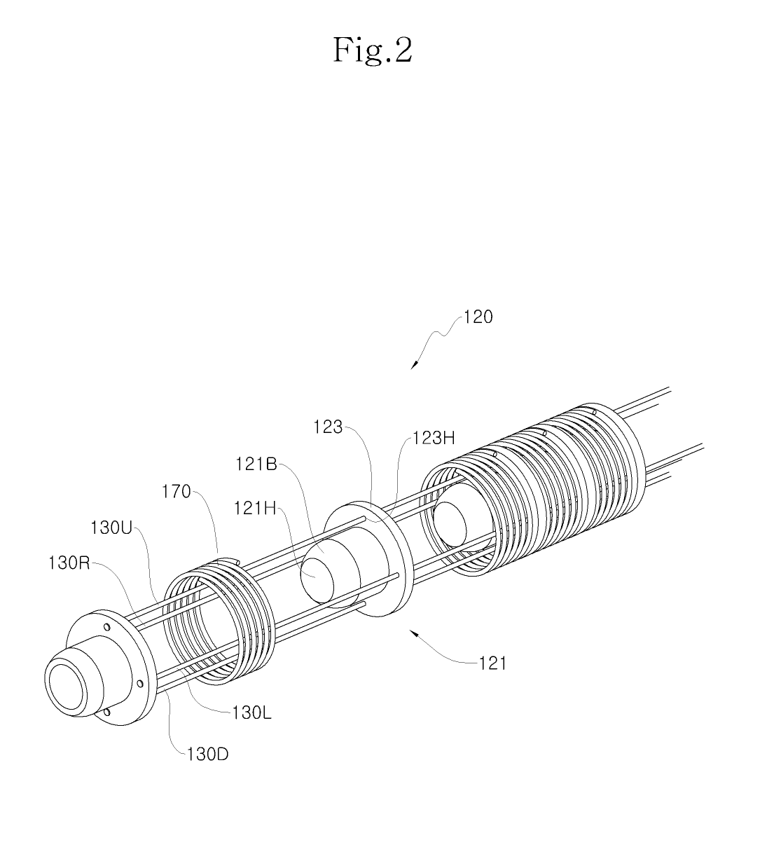

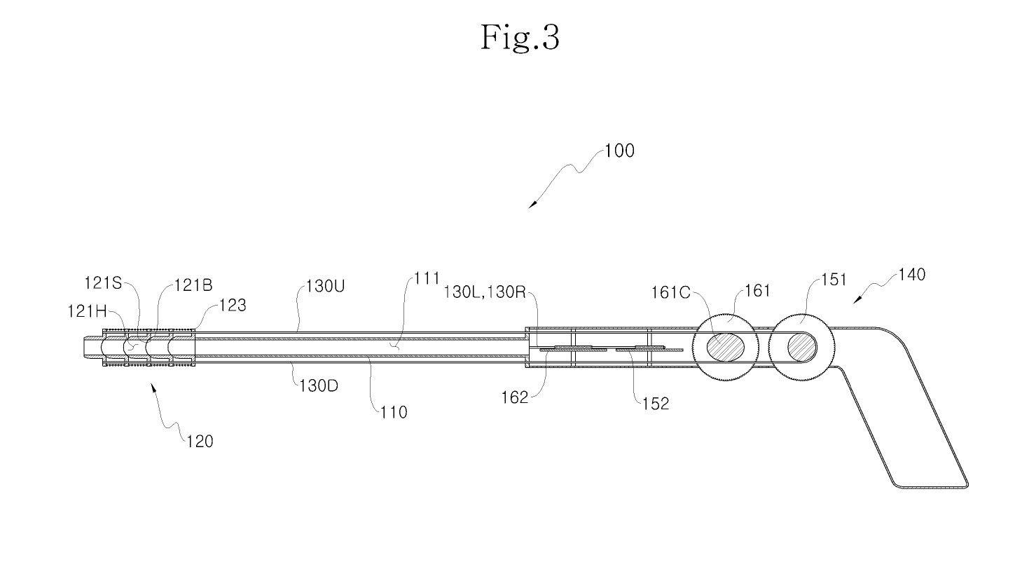

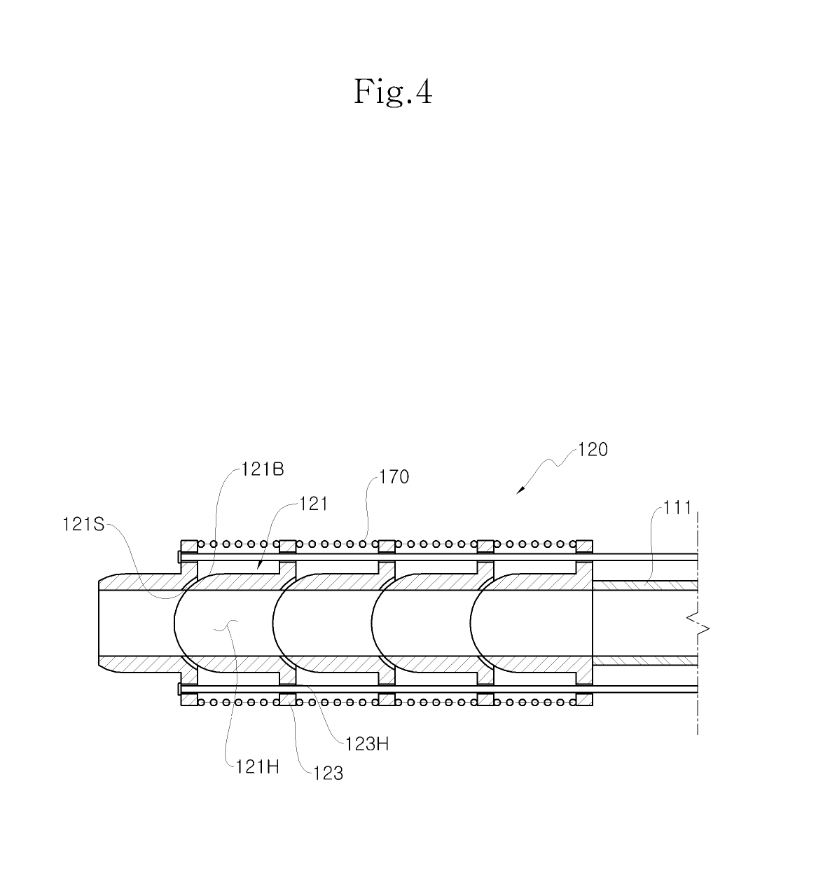

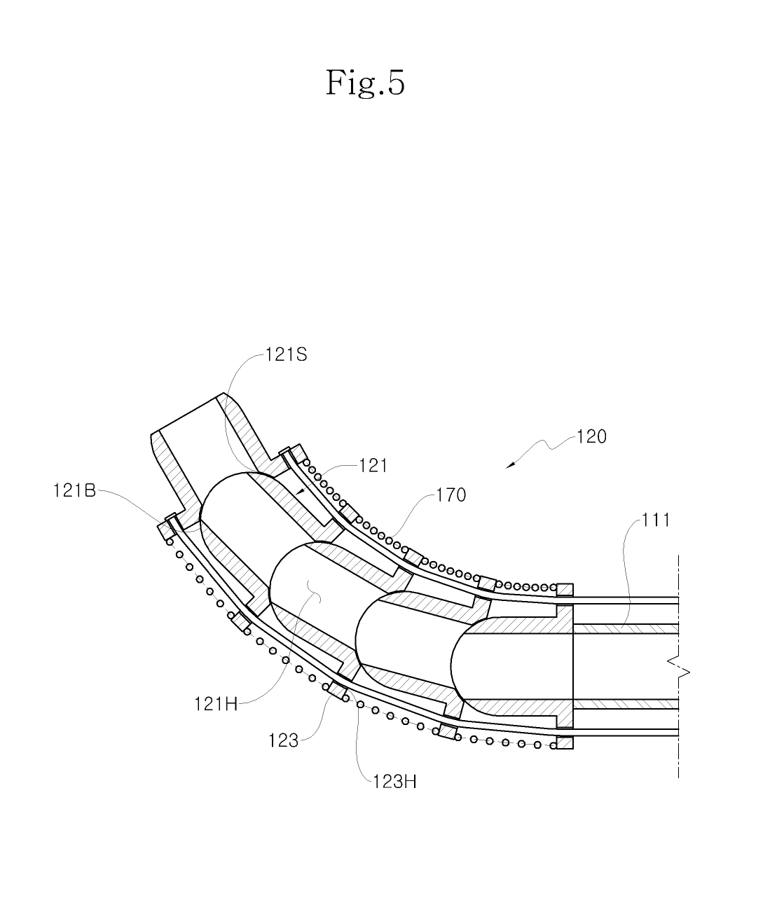

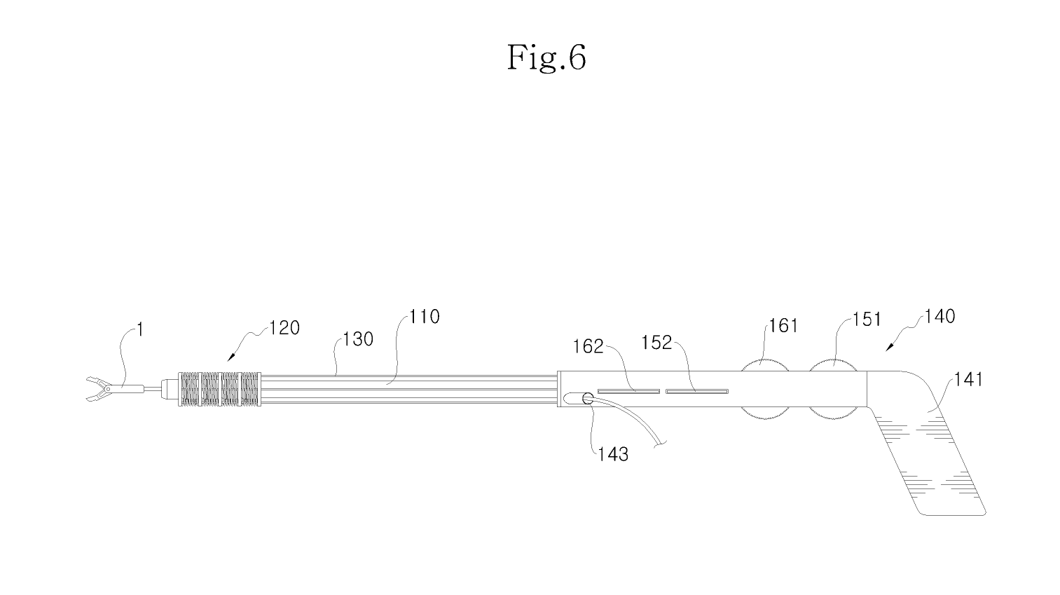

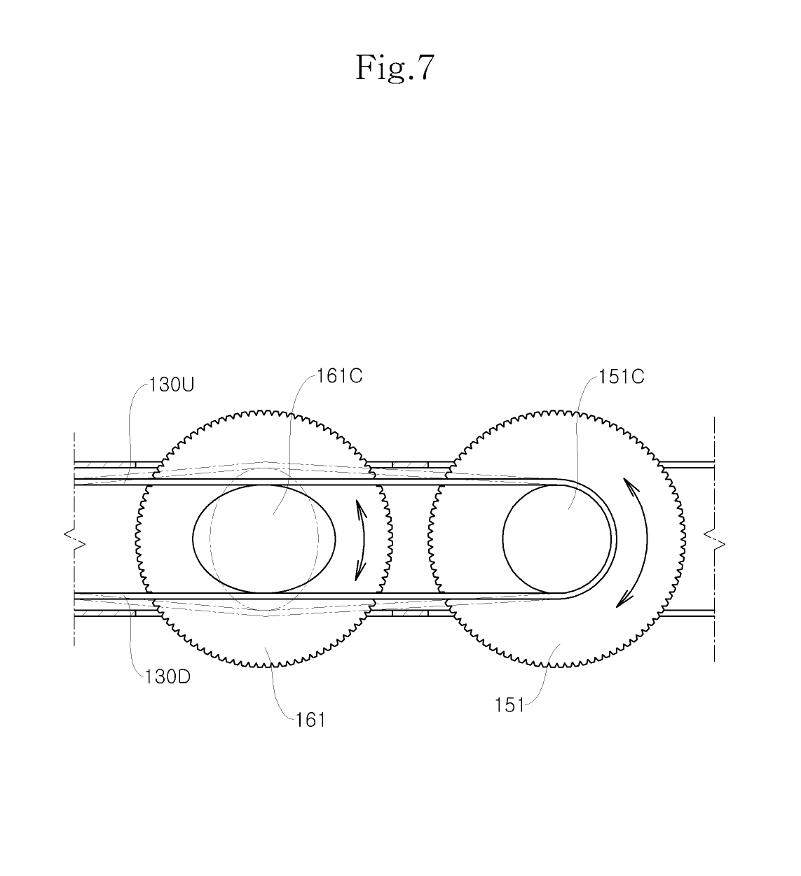

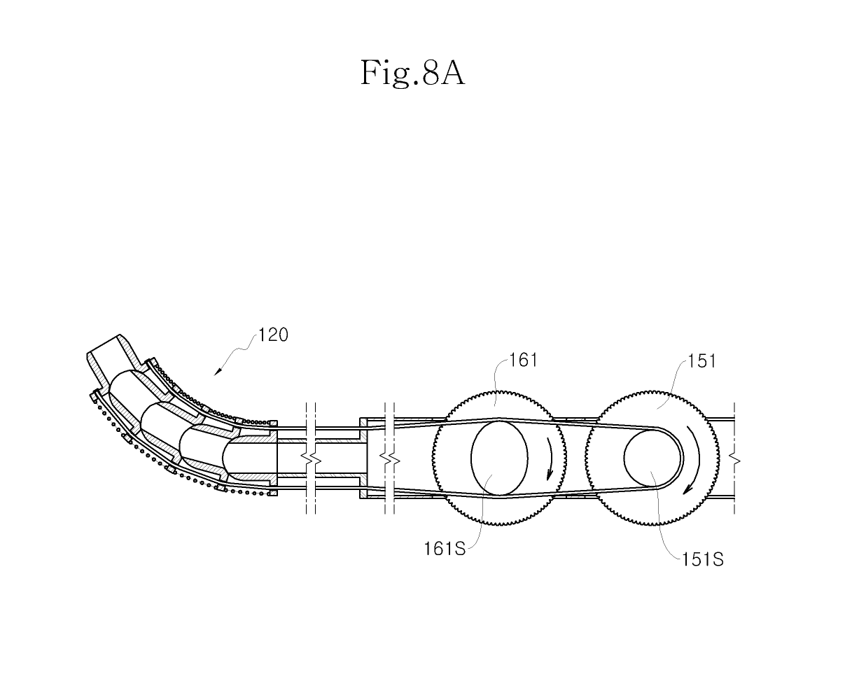

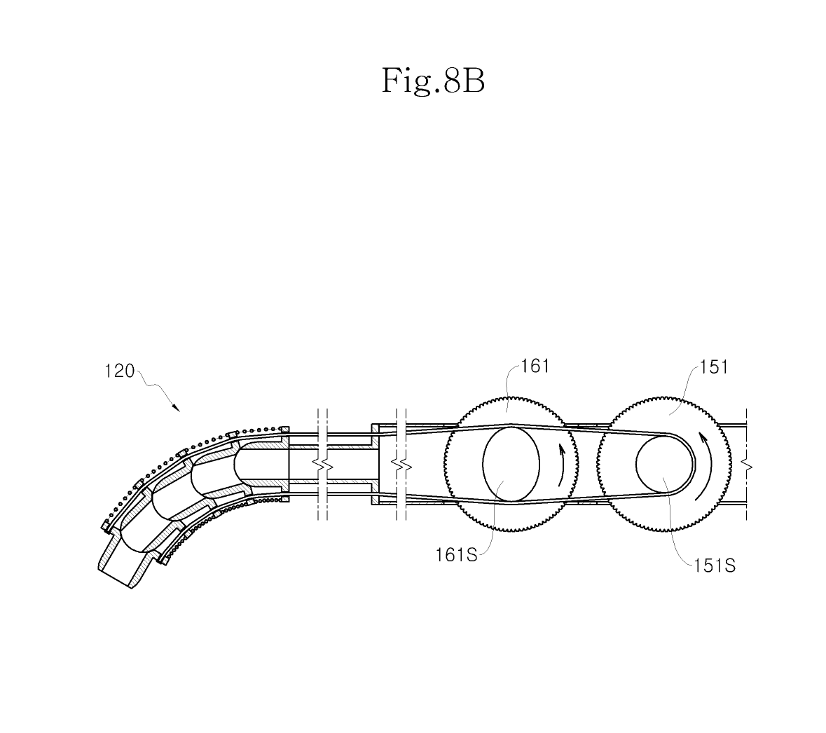

In the drawings, FIG. 1 is a perspective view showing a guide tube for microsurgical instruments according to the present disclosure, FIG. 2 is an exploded perspective view showing a terminal of the guide tube of FIG. 1, FIG. 3 is a cross-sectional view showing the guide tube of FIG. 1, FIG. 4 is a detailed diagram showing a coupling relation among a coil spring, a flange and a wire hole employed in the guide tube of FIG. 1, FIG. 5 is a cross-sectional view showing the terminal of the guide tube of FIG. 1 which is in a bending state, FIG. 6 is a conceptual diagram showing a microsurgical instrument inserted into the guide tube of FIG. 5, FIG. 7 is a detailed diagram showing a tension control unit employed in the guide tube of FIG. 1, and FIGS. 8A and 8B are conceptual diagrams showing a relation of controlling a curve and rigidity of the curved tube by using the tension control unit of FIG. 7.

As shown in FIG. 1, a guide tube 100 for microsurgical instruments includes a rigid linear tube 110 having a hollow formed therein in the length direction and a curved tube 120 having unit bodies 121 connected to the front end of the linear tube 110 by a ball-socket structure. Therefore, if the guide tube 100 for microsurgical instruments is used, the guide tube 100 is inserted to tissues of a human body, and the curved tube 120 is bent according to locations and conditions of the tissues so that the front of the curved tube 120 is oriented to the lesion. After that, a microsurgical instrument 1 such as a biopsy forceps is inserted and pushed through the hollow of the linear tube 110, the microsurgical instrument 1 may safely approach the lesion through the hollow of the linear tube 110 and the hollow of the curved tube 120.

Hereinafter, the guide tube for microsurgical instruments configured as above will be described in more detail.

The linear tube 110 is so rigid not to deform during a surgical operation. The linear tube 110 has a hollow 111 formed therein in the length direction and flanges 113 formed at the front and rear ends of the linear tube 110 to protrude outwards. Four holes 113H are formed in the flange 113 at 90 degrees, and the front end of the linear tube 110 connected to the curved tube 120 has a structure of a hemispherical ball 110B.

Meanwhile, as shown in FIGS. 2 to 4, the curved tube 120 has unit bodies 121 connected to each other so that the curved tube 120 may bent. The unit bodies 121 of the curved tube 120 have a cylinder structure with a hollow 121H. Here, the front end of the unit body 121 has a structure of the hemispherical ball 121B as described above, and the rear end of the unit body 121 has a structure of a socket 121S with a hemispherical groove.

As described above, the unit bodies 121 are located in a state where the ball 121B formed at the front end of a unit body 121 is matched with a socket 121S of a unit body 121 located at the front. Here, the coupling relation of the hemispherical groove and the hemispherical socket is called a ball-socket coupling structure, and the ball 110B formed at the front end of the linear tube 110 is also coupled to a socket 121S of a unit body 121 located at the rearmost end of the curved tube 120.

Meanwhile, a flange 123 is formed outwards in the middle of the length of the unit body 121, and four holes 123H are formed in the flange 123 at 90 degrees. In addition, a coil spring 170 is located to surround the outer circumferences of the unit bodies 121 connected by means of the ball 121B and the socket 121S, and both ends of the coil spring 170 are located to come into contact with the flanges 123 formed at the unit bodies 121 connected to each other. Here, the diameter of the coil spring 170 is smaller than the distance between the holes 123H located in the diameter direction of the flange 123. Therefore, the holes 123H are located out of the coil spring 170 surrounding the circumferences of the unit bodies 121.

In addition, the wire 130 passes through the hole 123H formed in the flange of the unit body 121. Here, the front end of the wire 130 is fixed to the flange 123H of the unit body 121 located at the front end of the curved tube 120, and the rear end of the wire 130 extends rearwards through the hole 113H of the flange 113 formed at the rear end of the linear tube 110.

The wire 130 extending to the rear of the linear tube 110 is connected to a tension control unit 140 which controls bending and rigidity of the curved tube 120. The tension control unit 140 will be described in detail later, and now the bending relation and the rigidity controlling relation of the curved tube 120 according to the displacements of the wires 130 will be described.

As shown in FIG. 3, if the tension of the wire 130 is loosened through the tension control unit 140, a gap between the outer circumference of the ball 121B and the inner circumference of the socket 121S increases in a state where the coupling structure of the ball 121B and the socket 121S is maintained by the elastic force of the coil spring 170. If the tension of the wire 130 is loosened as described above, the friction between the unit bodies 121 is weakened, and so the curved tube 120 may easily bend.

In order to bend the curved tube 120 in a state where the tension of the wire 130 is loosened, one or two wires located in the bending direction are pulled rearwards. If so, the unit bodies 121 of the curved tube 120 are bent as shown in FIG. 5. Here, if the bending direction is identical to the direction where the wires 130 are located, only one wire is pulled for bending. If the bending direction is not identical to the direction of the wires, two wires located at the bending direction are pulled to bend the curved tube 120 in a desired direction.

As the curved tube 120 bends as described above, the coil spring 170 also deforms in the bending direction so that an elastic force increases within the bending direction and relatively decreases out of the bending direction.

In a state where the curved tube 120 is bent by using one or two wires as described above, if four wires 130 are simultaneously pulled and tensed in order to enhance the rigidity of the curved tube 120, the frictional force of the curved tube 120 increases as the ball 121B and the socket 121S maintained in a bending state come into contact with each other. As described above, as the frictional force between the unit bodies 121 increases, the curved tube 120 maintains the bending state. In addition, even though a microsurgical instrument 1 entering through the hollow 111 of the linear tube 110 passes through the hollow 121H of the curved tube 120, the curved tube 120 may maintain its bending posture.

Meanwhile, in a case where the tensed wire 130 is released and maintained loose, the gap between the ball 121B and the socket 121S increases by the elastic force of the coil spring 170 like an initial state, thereby maintaining a bendable state.

Hereinafter, the tension control unit 140 for controlling a tension of a wire extending rearwards along the linear tube 110 will be described in detail.

As shown in FIGS. 7, 8A and 8B, the tension control unit 140 is mounted to the rear end of the linear tube 110. The tension control unit 140 includes a handle 141 formed at the rear end thereof, and a first steering dial 151 mounted to the rear end of the handle 141 to control tensions of two wires 130U, 130D located at upper and lower positions based on the cross-section of the linear tube 110. In addition, the tension control unit 140 includes a second steering dial 152 for controlling tensions of two wires 130L, 130R located at right and left positions based on the section of the linear tube 110, a first cam dial 161 for locking or releasing tensions of the two wires 130U, 130D located at the upper and lower positions, and a second cam dial 162 for locking or releasing tensions of the two wires 130L, 130R located at the right and left positions. As shown in FIG. 6, the tension control unit 140 has an insert hole 143 in which a microsurgical instrument 1 is inserted so that the microsurgical instrument 1 may enter the linear tube 110 and the curved tube 120.

In detail, the two wires 130U, 130D at the upper and lower positions, which advance rearwards along the linear tube 110, surround the first steering dial 151. Here, the two wires 130U, 130D may be configured with a single long wire, not divided, so as to be located at the upper and lower positions of the linear tube 110 while surrounding the first steering dial 151. In another case, the rear ends of divided two wires 130U, 130D may be fixed to the first steering dial 151.

According to the above configuration, when the first steering dial 151 turns, any one of the two wires 130U, 130D located at the upper and lower positions is loosened and the other is relatively tensed. As the tensions of the wires 130U, 130D located at the upper and lower positions of the linear tube 110 are controlled by the first steering dial 151, the curved tube 120 is bent upwards or downwards.

Meanwhile, the second steering dial 152 is located at right angle with the first steering dial 151, and the wires 130L, 130R located at the right and left positions of the linear tube 110 surround or fixed to the second steering dial 152, similar to the first steering dial 151 described above. Therefore, the curved tube 120 is belt left or right due to the turn of the second steering dial 152.

Therefore, by turning the first steering dial 151 and the second steering dial 152 together and thus controlling tensions of four wires 130U, 130D, 130L, 130R, the curved tube 120 may freely bend.

Meanwhile, a first cam dial 161 and a second cam dial 162 for locking or releasing the controlled tensions of the wires 130U, 130D, 130L, 130R are mounted to the tension control unit 140.

The first cam dial 161 is located at the front of the first steering dial 151 and includes a cam 161C located between the upper wire 130U and the lower wire 130D. If the first cam dial 161 is turned, the longitudinal shaft of the cam 161C is located in the vertical direction of the upper wire 130U and the lower wire 130D and pushes the upper wire 130U and the lower wire 130D outwards to be tensed integrally. If so, in a state where the curved tube 120 is bent due to the tension control of the first steering dial 151, the tensions of the upper wire 130U and the lower wire 130D gradually increase, and then the frictional force between the curved tube 120 and the unit body 121, namely between the ball 121B and the socket 121S, increases, which comes to the locked state. On the contrary, if the longitudinal shaft of the cam 161C is located parallel between the upper wire 130U and the lower wire 130D, the tension applied to the curved tube 120 is weakened, and the bending of the curved tube 120 is naturally released.

The second steering dial 152 and the second cam dial 162 also operate in the same principle as the first steering dial 151 and the first cam dial 161 described above. In other words, the tensions of the left wire 130L and the right wire 130R are controlled by the second steering dial 152, and in a state where tension is controlled, the bending is locked or released by the turn of the second cam dial 162.

Meanwhile, even though the guide tube 100 has been described in a way that the curved tube 120 is bent, locked and lock-released by means of four wires 130U, 130D, 130L, 130R located at the circumferences of the curved tube 120 and the linear tube 110, it is also possible that only the left wire 130L and the right wire 130R are provided so that the curved tube 120 is bent, locked and lock-released only in the right and left direction. In addition, it is also possible that only the upper wire 130U and the lower wire 130D are provided so that the curved tube 120 is bent, locked and lock-released only in the upper and lower directions.

While the exemplary embodiments have been shown and described, it will be understood by those skilled in the art that various changes in form and details may be made thereto without departing from the spirit and scope of the present disclosure as defined by the appended claims.

What is claimed is:

1. A guide tube for microsurgical instruments, comprising:
a linear tube having a hollow formed in the length direction thereof;
a curved tube mounted to the front end of the linear tube and having unit bodies connected to allow bending;
an elastic body for giving an elastic force so that a gap between the unit bodies increases; and
a tension control unit mounted to the rear end of the linear tube to bend the curved tube by controlling tensions of a plurality of wires extending from the curved tube, wherein the tension control unit controls the tensions of the wires in a state where the curved tube is curved so that the rigidity of the curved tube increases to support a surgical instrument located in the linear tube and the curved tube.

2. The guide tube for microsurgical instruments according to claim 1, wherein the unit body of the curved tube has a hemispherical ball formed at a front end thereof and a socket with a hemispherical groove formed at a rear end thereof, and the ball of the unit body is matched with and connected to a socket of another unit body.

3. The guide tube for microsurgical instruments according to claim 2, wherein a flange is formed at the outer circumference of the unit body, and a wire having one end fixed to the unit body located at the front end of the curved tube extends rearwards through a hole formed in a flange of another unit body.

4. The guide tube for microsurgical instruments according to claim 3, wherein the elastic body is a coil spring, which is located at the inside of the wire passing through the hole of the flange while surrounding the outer circumference of the unit body, and both ends of the coil spring come into contact with flanges of unit bodies located in the front and rear direction.

5. The guide tube for microsurgical instruments according to claim 2, wherein the front end of the linear tube has a hemispherical ball structure, which is matched with a socket of a unit body located at the rear end of the curved tube.

6. The guide tube for microsurgical instruments according to claim 5, wherein a flange is formed at the outer circumference of the linear tube, and a wire extending rearwards from the curved tube extends to the tension control unit through a hole formed in the flange of the linear tube.

7. The guide tube for microsurgical instruments according to claim 1, wherein a plurality of wires disposed at equal angles along the circumferences of the linear tube and the curved tube extend in the length direction of the curved tube and the linear tube and are connected to the tension control unit.

8. The guide tube for microsurgical instruments according to claim 7, wherein there is provided a pair of tension control units, which contrarily control tensions of the wires extending to the rear of the linear tube so that the curved tube is bent.

9. The guide tube for microsurgical instruments according to claim 7, wherein there is provided a pair of tension control units, which increase or decrease tensions of the wires extending to the rear of the linear tube in a lump so that the rigidity of the curved tube is controlled.

10. The guide tube for microsurgical instruments according to claim 8, wherein the tension control unit includes a steering dial connected to the rear ends of the wires to contrarily control tensions of the wires by turning.

11. The guide tube for microsurgical instruments according to claim 9, wherein the tension control unit includes a cam dial for locking the curved tube to maintain a curved state by increasing a gap between the wires extending rearwards and increasing tensions of the wires or releasing the locking of the curved tube so that the curved tube is capable of bending by decreasing the gap between the wires and decreasing the tensions thereof.

12. The guide tube for microsurgical instruments according to claim 10, wherein there are provided two wires, which are respectively located at both sides of the linear tube and the curved tube, and tensions of two wires are contrarily controlled by turning the steering dial connected to the rear ends of the two wires.

13. The guide tube for microsurgical instruments according to claim 11, wherein there are provided two wires, which are respectively located at both sides of the linear tube and the curved tube, and a cam of the cam dial located between two wires is turned to increase a gap between the two wires and increase tensions of the two wires or decrease the gap between the two wires and decrease tensions of the two wires.

14. The guide tube for microsurgical instruments according to claim 10, wherein there are provided four wires, which are located at equal angles along the circumferences of the linear tube and the curved tube, and tensions of two wires are contrarily controlled by turning a first steering dial connected to the rear ends of the two wires and tensions of the other two wires are contrarily controlled by turning a second steering dial connected to the rear ends of the other two wires.

15. The guide tube for microsurgical instruments according to claim 11,
wherein there are provided four wires, which are located at equal angles along the circumferences of the linear tube and the curved tube, and a cam of a first cam dial located between two wires is turned to increase a gap between the two wires and increase tensions of the two wires or decrease the gap between the two wires and decrease tensions of the two wires, and
wherein a cam of a second cam dial located between the other two wires is turned to increase a gap between the other two wires and increase tensions of the other two wires or decrease the gap between the other two wires and decrease tensions of the other two wires.

16. The guide tube for microsurgical instruments according to claim 1, wherein an insert hole is formed in the tension control unit so that a surgical instrument is inserted into the hollow of the linear tube and the hollow of the curved tube.
